(19) United States
(12) Patent Application Publication
(10) Pub. No.: US 2005/0121041 A1
(43) Pub. Date: Jun. 9, 2005

(54) Human extremity restraint

(76) Inventor: David A. R. Barnitz, Rio Linda, CA (US)

Correspondence Address: Mark C. Jacobs, Esq., 3033 El Camino Avenue, Sacramento, CA, 95821-6014, US

(21) Appl. No.: 10/730,315

(22) Filed: Dec. 4, 2003

(51) Int. Cl. A61B 19/00

(52) U.S. Cl. 128/879; 128/869; 128/878

(57) ABSTRACT

A restraint for application to an extremity of a wearer instead of metal or plastic cuffs. The device has a main body and a series of bands, which bands overlay a transversely disposed webbing section and extend beyond the body portion. The body is formed of two layers, a tough upper layer of canvas or similar material and a soft bottom layer such as velour that makes skin contact. The body is generally rectangular and preferably has an oblique inner edge that connects the parallel upper and lower edges. Bands of hook and pile closure are disposed along the width of the body between the outer edge and the preferably oblique edge and extend an equal amount beyond the body. These bands have pile disposed away from the body, and the band part that extends beyond the body has a pile layer obverse side and a hook layer reverse side.

### FIELD OF THE INVENTION

This application relates to a device to be used primarily at psychiatric and medical facilities as a restraining means for inhibiting movement of patients without significant discomfort. It may also be useful for other applications requiring nonabrasive restraints.

### BACKGROUND OF THE INVENTION

Restraints are used by psychiatric and medical personnel to restrain out-of-control persons under a variety of circumstances and may also be used in other various operating environments.

Restraints are also used by hospital attendants, nurses, EMT's, and others to prevent persons from doing harm to themselves, other persons and to a property, at a particular point in time.

One of the problems associated with current restraint apparatuses is that if applied too tight, they can be injurious by causing skin abrasions, blood flow constriction and other problems to the wearer. If too loose, the wearer might be able to escape and cause injury to himself or others. Therefore, there is a need for a restraint that is non injurious to the wearer. This invention fills that need by providing a strong and effective, fabric-based, restraining device that is strong enough to restrain the wearer, and which is noninjurious and causes minimal discomfort.

The invention accordingly comprises the device possessing the features, properties, the selection of components which are amplified in the following detailed disclosure, and the scope of the application of which will be indicated in the appended claims.

For a fuller understanding of the nature and objects of the invention, reference should be made to the following detailed description, taken in conjunction with the accompanying drawings.

### SUMMARY OF THE INVENTION

A fabric-based restraint to detain, bind, restrict or hinder the movement of the wearer. The device may be applied to the arms or legs of any size, for both children and adults without harming the wearer. The wraparound unit includes a built-in formed central loop to allow attachment of a location inhibitor, such that the device wearer can be secured in a fixed position such as a bed or Gurney.

The device of this invention may also be attached wrist to wrist or ankle to ankle for mobile restraint.

It is a first object to provide a fabric-based restraining device.

It is a second object to provide a restraining device that has an interchangeable throwaway skin contacting layer.

It is a third object to provide a washable reusable nonabrasion causing restraint.

Other objects of the invention will in part be obvious and will in part appear hereinafter.

### BRIEF DESCRIPTION OF FIGURES

FIG. 1 is a perspective view of this invention on the clothed wrist of a wearer FIG. 2 is a top plan view of this device.

FIG. 3 is a bottom plan view of this invention.

FIG. 4 is a top plan view of a variant having only two securing bands.

FIG. 5 is a perspective view of a removable and disposable pad insert that can be utilized as part of this invention shown in exploded view, and showing the adaptation required for use.

FIG. 6 is a bottom plan view of the inventive device modified to receive the pad insert that forms part of this invention.

### DESCRIPTION OF THE PREFERRED EMBODIMENT

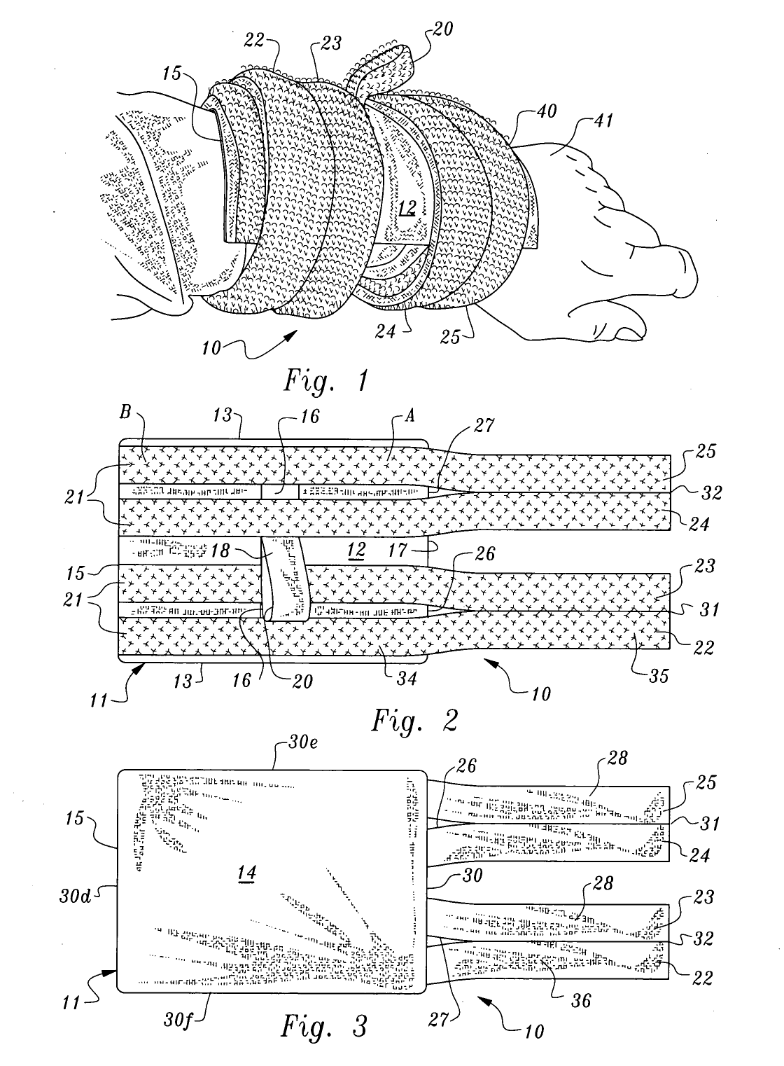

In FIG. 1 the device 10 of this invention is seen wrapped around the clothed arm of the wearer. Loop 20 which is built in and centrally disposed and which will be discussed infra in detail is used to attach the wearer to a wall, chair or bedframe as by a shackle, chain or rope. The discussion now moves to FIG. 2.

The device of this invention is seen laid out in unrolled form in FIGS. 2 and 3. The device has a main body 11 and a series of bands 21, which bands preferably overlay a transversely disposed webbing section or segment 18.

Device 10 has a main body 11 formed of two layers, an outer layer 12 which may be of cotton, polyester, nylon or some other close woven material such as canvas. The inner layer 14 of the body 11 that lies upon the skin is a soft layer of fabric such as but not limited to velour. The main body has upper and lower parallel edges, 19, an inner oblique edge 17 and an outer edge 15. The outer edge 15 is disposed at a substantially right angle to the upper and lower edges.

There are four bands 22,23,24 and 25 that are attached to the upper layer 12 of the main body 11. These four bands are spaced apart from top to bottom and extend the full width of the body from the outer edge 15 to the oblique edge 17. While being spaced apart, they converge in pairs at points spaced slightly beyond the oblique edge of the body. The two inner bands 23,24 are disposed in a convergent fashion toward each of the respective two outer bands at the oblique or inner edge, 17, of the body 11. A notch 26 and a notch 27 are formed at the junction of the respective two bands, approximately one inch beyond the inner edge 17, of the body portion. The bands could converge along the length of the body as well. While such is not shown, this is deemed to be within the skill of the artisan to do so.

From the points of convergence each of the two bands that form a pair namely 22 and 23 and the pair 24 and 25 are sewn together along seam 31 for the pair joined at notch 26 namely the pair now known as 22-23. The pair of bands 24 and 25 joined at notch 27 are also seamed together long their facing edge to form the pair now known as 24-25.

Returning briefly to the placement of the bands on the body 11, it is seen that the spacing between band 23 and band 24 need not conform to the spacing between the two members of the respective pairs 22 and 23 & 24 and 25. Band 22 and band 25 should preferably be disposed along the upper and lower edges of the outer fabric layer 12. Again see FIG. 2. The stitch to be used for the sewing is preferably a blind stitch so that a wearer cannot try to break the thread using any type of implement available to him or her.

The “A” section of each band—that part that extends beyond the edge of the body is equal in length to the “B” section that is attached to the body 11. The reason for the equality of length of the bands is such that when they are mated, the overlap will be co-extensive.

Each of the separate bands 22,23,24, and 25 for their “A” section, has only a top surface 34 which is of the pile material of the closure. The “B” section of each band also has a pile layer 35 on the top side, and a hook layer 36 of Velcro® or equal on the underside of the band.

Disposed transverse to the running of the four bands is a section of webbing, usually of nylon, which is readily available in the marketplace. This length of webbing 16 is cut to a size larger than the extension between the upper and lower edges of the main body 11. The webbing is sewn spaced to the body commencing at the upper and lower edges of the body at a location near or at the midpoint of the width of the body, with the outer edges of the webbing being at substantially the upper and lower edges of the body. That mode of attachment leaves a raised up or loop portion 18 in the middle which loop is spaced from the main body. The loop 18 has an opening therein designated 20. It is through this loop 18 that a chain or shackle or handcuff or other confining tool can be used to limit the ability of the wearer to move away, or to touch a part of the body with the retained extremity. See FIG. 2. A cable tie, readily available to the art, can be attached to the loop of a pair of these restraints to secure a person's hands together during a crowd control action. While sewing the webbing to the body before the addition of the bands is preferred, either step can take place first.

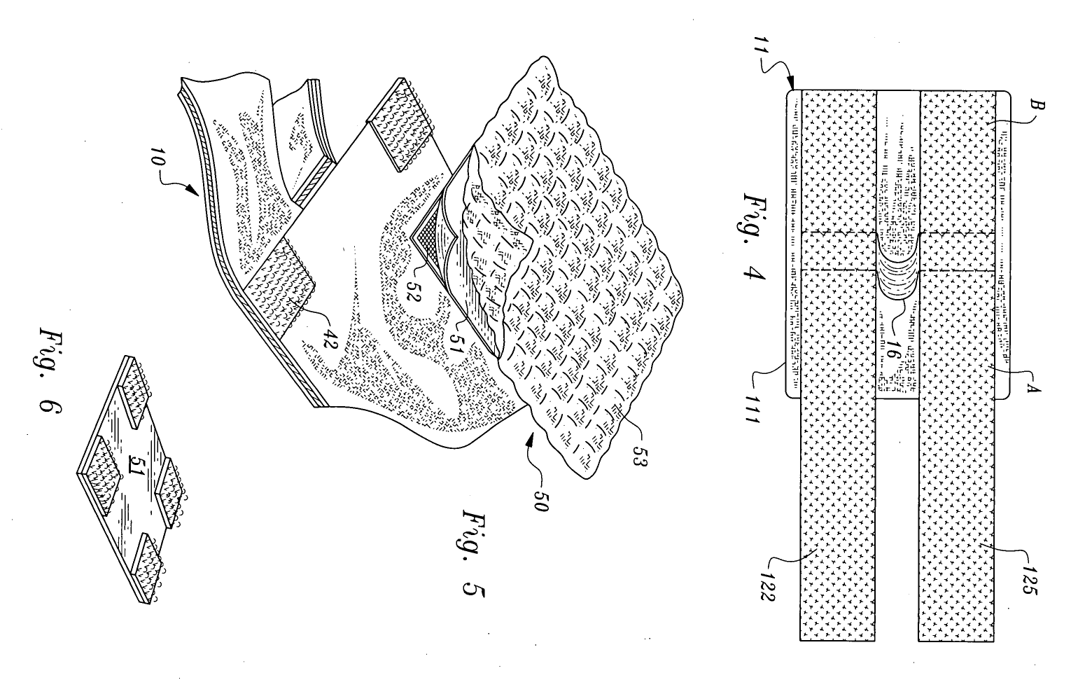

The device of the first embodiment was made with four bands of about one inch in elevation that converged together as to form two bands of a height equal to two individual bands. The embodiment is practical and uses one inch wide bands of Velcro° brand hook and pile closure, which is readily available in several colors. But in point of fact only two bands are needed if two inch wide hook and pile closure is available. Reference is thus made to FIG. 4 wherein like numbers refer to like parts. One band 122 is sewn to the body 11 adjacent the upper edge, and one band 125 adjacent the lower edge of the body 111 both overlying the webbing section 16. Here too, the “A” portion of the two bands is of loop material only, while the “B” aspect is both loop on the obverse side and hook on the reverse side.

Preferably the upper and lower edge seams that connect the canvas or corduroy fabric of the upper layer 12 to the lower layer of velour, velvet, etc, is ripple layered along the length of the upper and lower edges to prevent bunching.

While the discussion previously has placed the oblique edge of the body proximal the two respective notches seen in FIGS. 2 and 3, the oblique edge could also be the outer or distal edge if desired.

In FIG. 5 there is shown the optional pad insert 50. It has a flexible film base 51 of waterproof paper, polyester, or nylon to which is attached in each corner a Velcro® or equal tab 52 on the underside of the base 51. See FIG. 6. A pad 53 of woven or nonwoven fabric or even cotton batting is removably, adhesively, attached preferably to te film base 51's top side. The pad may be fixedly attached but his renders cleaning and replacement for sanitary reasons more difficult.

The pad is worn adjacent the skin. The film base 51 is attached by its tab 52 to a matingly engageable pad of Velcro® on the underside of the invention 10. The pad is worn adjacent the skin such that abrasions to the wrist and/or ankle can be minimized or eliminated altogether.

The use of the insert pad 50 is optional, but is beneficial in the fight against disease transmission from wearer to wearer. In the medical field, concerns of cross contamination and universal precautions require that restraints be sufficiently cleaned between patients. A disposable pad attached to the restraint at the contact point with the skin's surface of the wearer will minimize cross contamination and the necessary cleansing of the body of the restraint. This disposable pad can be made of nonirritating paper or cloth such as felt so that the paper or felt can absorb fluid without irritating the skin and has a water-resistant backing to prevent body fluid from leaking into the restraint.

If the detachable pad 50 is not used, the surface 14, which contacts the wearer's skin, may be of soft, nonirritating, washable, permanently affixed, felt-like material.

For ease and convenience, the film base is generally rectangular in configuration with a Velcro® tab in each of the four corners' underside.

It is seen that I have devised an article that can be washed and reused many times, such as between uses. If there is a possibility that the most recent wearer of the device may be infected with HIV or a sexually transmitted disease, immediate disinfection can be carried out. Unlike metal handcuffs, the restraint of this invention forms no potential weapon for the wearer against the hospital, jail or other facility personnel nearby.

It is coincidental that the timing of this patent application arises close to the time of recall of 1,000 sets of handcuffs used by the sheriff's department in Los Angeles County California for fear of hurting suspects' wrists and the desire not to create inadvertent injures, according to a recent Associated Press article. This device, especially when used with the optional insert pad, totally avoids that issue.

It is contemplated to have the device sized, small, medium, large and extra large to fit both hands and wrists of people of all ages and builds. The sizing would be in the body of the unit by making the distance between the upper and lower parallel edges greater or lesser than the medium size general fit model, which is of a distance of about six inches. The spacing between the outer edge and the oblique edge can also be modified from the medium sized model of about the outer edge and the oblique edge can also be modified from the medium sized model of about nine inches within the range of six to twelve inches. Thus, the medium model has bands of about 18 inches in length for both the “A” and the “B” portions. A loop of about two inches is considered adequate. Color coding can be readily accomplished if desired as by gender, size or special use high infection risk

The device of this invention can be readily used in an operating recovery room as well as by law enforcement. Being of fabric it can be readily sterilized. Being lightweight a plurality can be stacked in boxes for quick use during protests and other crowd control situations, by simply cuffing a person and chaining or shackling the extremity bearing this device to a wall, fence or other fixed object. Two of the devices can be attached via the loop using simple cable tie where two hand restraint is deemed necessary. By being fabric no injury to the wearer will occur, thus avoiding lawsuits for excessive force or injury to the party upon whom the device or devices is/are placed.

In extremely dangerous situations where dangerous or violent individuals are involved and where escape may be of concern, a locking handcuff or similar device may be applied over the invention without compromising any of the invention's virtues.

Since certain changes maybe made in the described apparatus without departing from the scope of the invention herein involved, it is intended that all matter contained in the above description and shown in the accompanying drawings shall be interpreted as illustrative and not in a limiting sense.

1. A handcuff replacement restraint for arms or legs comprising a body portion having an upper layer and a lower layer,
said body portion having a generally rectangular body with parallel upper and lower edges, and an outer edge normal to said upper and lower edges, but preferably having the inner body edge slightly oblique,
said body having a transversely disposed segment of webbing secured to said body at the termini of the band, and being formed into a central loop spaced from said body,
a plurality of bands each of which has an “A” and “B” segment,
said “A” segment being loop pile closure secured along the length of the body from the outer edge to the inner edge thereof, the loop surface disposed outwardly from said body,
the “B” segment extending from the inner edge of said body a distance equal to the body length,
said “B” segment having a loop pile upper surface, and a hook fabric closure lower surface.
whereby when the body portion is wrapped around a body limb, and the “B” segment of the band is overlaid on the “A” segment, the limb can be restrained by attachment of a chain, rope, belt or the like to the loop and to a fixed object to prevent movement of the wearer.

2. The device of claim 1, wherein there are two hook and pile closure bands attached to the body and extending outwardly therefrom.

3. The device of claim 1, wherein there are four hook and pile closure bands attached to the body, each spaced slightly from each other, and wherein each adjacent pair of bands is sewn together along the adjacent edge between the first pair and along the adjacent edge between the second pair, commencing at a point along the length of each respective band.

4. The device of claim 3, wherein the webbing segment is disposed beneath the bands and an upstanding loop is centrally disposed between the second and third bands.

5. The device of claim 1, wherein the body upper layer is canvas and the bottom layer is velour.

6. The device of claim 1, wherein the webbing segment is nylon.

7. The device of claim 4, wherein the webbing segment is nylon.

8. The device of claim 3, wherein the seamed junction of each of the two pairs of bands The device of claim 1, wherein the webbing segment is about 1 inch wide and is disposed beneath the bands of closure, said webbing segment having a central loop of about 2 inches.

9. The device of claim 1, wherein the webbing segment is about 1 inch wide and is disposed beneath the bands of closure, said webbing segment having a central loop of about 2 inches.

10. A replaceable pad insert for use in a fabric-based restrainer, said insert comprising:
a flexible film base having a tab side and an underside; and having a plurality of front Velcro® tabs on the underside thereof, and a pad adhesively attached to the top side of the film base.

11. The replaceable pad of claim 10, wherein the film base is rectangular, and there is a second Velro® or equal tab on each corner of the underside of the film base.

12. The combination of handcuff replacement restraint of claim 1, further including a plurality of front Velcro® tabs on the underside of the body portion, and a replaceable pad insert mountable to the underside of said restraint, said pad insert having a flexible film base with a series of second Velcro® tabs on a first side thereof; and an adhesively attached pad attached to a second side of said film base; said first and second Velcro® tabs being removably connected to each other.

13. The combination of claim 12, wherein the pad is selected from woven and nonwoven fabric, and cotton batting.

14. The combination of claim 11, wherein the film base is rectangular having four second Velcro® tabs, one being in each corner.

15. The combination of claim 14, wherein the film base is selected from films of nylon, polyester, and waterproof paper.

16. The combination of claim 15, wherein the pad is removably adhesed to the film base.
